(19) United States
(12) Patent Application Publication
(10) Pub. No.: US 2010/0056644 A1
(43) Pub. Date: Mar. 4, 2010

(54) PHARMACEUTICAL COMPOSITIONS CONTAINING ANHYDROUS CALCIPOTRIENE

(76) Inventors: Nilendu Sen, Navi Mumbai (IN); Kusum Gole, Maharashtra (IN); Amol Mandhare, Maharashtra (IN)

Correspondence Address: GLENMARK PHARMACEUTICALS INC USA, 750 CORPORATE DRIVE, MAHWAH, NJ, 07430, US

(21) Appl. No.: 12/515,984

(22) PCT Filed: Nov. 28, 2007
(86) PCT No.: PCT/IB07/03662

§ 371 Date: May 22, 2009

Related U.S. Application Data
(60) Provisional application No. 60/873,876, filed on Dec. 8, 2006.

(30) Foreign Application Priority Data
Nov. 29, 2006 (IN) 1964/MUM/2006

(51) Int. Cl. A61K 31/045; A61P 17/06

(52) U.S. Cl. 514/729

(57) ABSTRACT

A stable pharmaceutical composition in a semisolid dosage form comprising a therapeutic effective amount of solubilized calcipotriene in an anhydrous form is disclosed.

### PRIORITY

This application claims the benefit under 35 U.S.C. §119 to U.S. Provisional Application No. 60/873,876, filed on Dec. 8, 2006, and entitled “PHARMACEUTICAL COMPOSITIONS CONTAINING ANHYDROUS CALCIPOTRIENE” and to Indian Provisional Application No. 1964/MUM/2006, filed on Nov. 29, 2006, and entitled “PHARMACEUTICAL COMPOSITIONS CONTAINING ANHYDROUS CALCIPOTRIENE”, the contents of each of which are incorporated by reference herein.

### BACKGROUND OF THE INVENTION

1. Technical Field

The present invention generally relates to a stable topical pharmaceutical composition containing anhydrous calcipotriene and process for its preparation.

2. Description of the Related Art

Calcipotriene, also known as (1R,3S)-5-[2-[(1R,3aR,7aS)-1-[(2S)-5-cyclopropyl-5-hydroxy-pent-3-en-2-yl]-7a-methyl-2,3,3a,5,6,7-hexahydro-1H-inden-4-ylidene]ethylidene]-4-methylidene-cyclohexane-1,3-diol or calcipotriol, is represented by the chemical structure of Formula I:

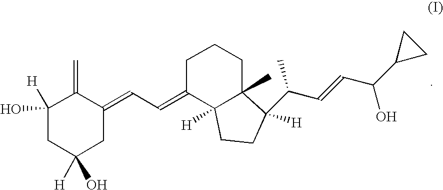

Calcipotriene is a topical medication used for the treatment of psoriasis, marketed under the trade name Dovonex®. Calcipotriene has minimal side effects and can be used over the short- or long-term. Calcipotriene is a vitamin-D derivative, about 1% as powerful as the natural hormone calcitriol (also known as 1,2,5 dihydroxycholecalciferol). Calcipotriene, which contains vitamin D₃, controls the rapid growth of skin cells. Calcipotriene is indicated for the treatment of psoriasis. It works by controlling the overproduction of skin cells in areas affected by psoriasis. See, e.g., The Merck Index, Thirteenth Edition, and Physician's Desk Reference, “DOVONEX”, 60th Edition, pp. 3374 (2006).

U.S. Pat. No. 5,292,727 (“the '727 patent”) discloses the use of certain Vitamin D analogues in the preparation of a pharmaceutical preparation for the treatment of acne. The '727 patent further discloses a method of treating acne which comprises administering to a subject in need of such treatment an effective amount of calcipotriol.

U.S. Pat. No. 5,763,426 (“the '426 patent”) discloses that a crystalline bulk drug is usually subjected to micronization or to a wet milling process in order to reduce the crystal size before the final suspension formulation is prepared. The '426 patent further discloses that it is technically difficult to perform a wet ball milling process when using the anhydrous crystal form of calcipotriene described in WO 87/00834. This problem was overcome by using a new crystalline form of calcipotriol, i.e., calcipotriol hydrate, instead of the anhydrous form.

Accordingly, there remains a need for improved pharmaceutical compositions containing calcipotriene.

### SUMMARY OF THE INVENTION

In accordance with one embodiment of the present invention, a stable pharmaceutical composition in a semisolid dosage form is provided comprising a therapeutic effective amount of calcipotriene in anhydrous form.

In accordance with a second embodiment of the present invention, a stable pharmaceutical composition in a semisolid dosage form is provided comprising a therapeutic effective amount of calcipotriene in an anhydrous form wherein the ratio of anhydrous calcipotriene:water is not more than about 1:100.

In accordance with a third embodiment of the present invention, a stable pharmaceutical composition in a semisolid dosage form is provided comprising a therapeutic effective amount of calcipotriene in anhydrous form, wherein the dosage form is in the form of cream.

In accordance with a fourth embodiment of the present invention, a stable pharmaceutical composition in a semisolid dosage form is provided comprising a therapeutic effective amount of calcipotriene in anhydrous form, wherein the pharmaceutical composition contains less than about 0.5% of a single maximum unknown impurity at about 40° C. and about 75% relative humidity after three months.

In accordance with a fifth embodiment of the present invention, a stable pharmaceutical composition in a semisolid dosage form is provided comprising a therapeutic effective amount of calcipotriene in an anhydrous form wherein the anhydrous calcipotriene is in solubilized form.

In accordance with a sixth embodiment of the present invention, a stable pharmaceutical composition in a semisolid dosage form is provided comprising a therapeutically effective amount of calcipotriene in an anhydrous form wherein the anhydrous calcipotriene is solubilized either in oil or non-aqueous water soluble solvent.

In accordance with a seventh embodiment of the present invention, a stable pharmaceutical composition in a semisolid dosage form is provided comprising a therapeutically effective amount of calcipotriene in anhydrous form, wherein the dosage form is in the form of cream containing anhydrous calcipotriene in an amount of about 0.001 to about 0.1% w/w.

In accordance with an eighth embodiment of the present invention, a process for preparing a stable pharmaceutical composition in a semisolid cream dosage form is provided comprising: (a) preparing an oil phase containing solubilized calcipotriene in an anhydrous form; (b) preparing an aqueous phase containing one or more buffering agents; (c) adding the oil phase of step (a) to the aqueous phase of step (b); and (d) emulsifying and homogenizing the product of step (c).

In accordance with a ninth embodiment of the present invention, a process for preparing a stable pharmaceutical composition in a semisolid cream dosage form is provided comprising: (a) preparing an oil phase containing solubilized calcipotriene in an anhydrous form; (b) preparing an aqueous phase containing one or more buffering agents and solubilized calcipotriene in an anhydrous form; (c) adding the oil phase of step (a) to the aqueous phase of step (b); and (d) emulsifying and homogenizing the product of step (c).

In accordance with a tenth embodiment of the present invention, a process for preparing a stable pharmaceutical composition in a semisolid cream dosage form is provided comprising: (a) dissolving calcipotriene in propylene glycol; (b) adding ethylenediaminetetraacetic acid (EDTA) and disodium phosphate dehydrate to water; (c) adding the product of step (a) to the product of step (b) and heating the mixture to 50-55° C.; (d) forming an oil phase comprising melting polyethylene glycol-400, polyethylene glycol-3350, glyceryl stearate/PEG 100 stearate, glyceryl monostearate and adding octyldodecanol; (e) adding the mixture of step (c) to the oil phase of step (d); (f) adding aluminum starch octenylsuccinate and titanium dioxide to the product of step (e) and homogenizing for a sufficient time; and (g) cooling under stirring at room temperature to form a cream.

In accordance with an eleventh embodiment of the present invention, a method of treating psoriasis in a human subject is provided comprising topically administering a pharmaceutical composition comprising a therapeutically effective amount of calcipotriene in an anhydrous form in a suitable dosage form.

### DEFINITIONS

The term “topical pharmaceutical composition” refers to a composition that is employed to prevent, reduce in intensity, cure or otherwise treat a target condition or disease.

The terms “topical” or “topical administration” or “local” are used in its conventional sense to mean delivery of a topical drug or pharmacologically active agent to the skin or mucosa. Topical administration, in contrast to transdermal administration, provides exclusively or predominantly a local rather than a systemic effect. The term “transdermal” is intended to include “transmucosal” drug administration, i.e., administration of a drug to the mucosal (e.g., sublingual, buccal, vaginal, rectal) surface of an individual so that the drug passes through the mucosal tissue and into the individual's blood stream.

The term “subject” or “a patient” or “a host” as used herein refers to mammalian animals, preferably human.

The terms “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The terms “effective amount” or “therapeutically effective amount” of a pharmacologically active agent is meant a non-toxic but sufficient amount of the drug or agent to provide the desired effect. The “effective amount” will vary depending on the compound, the disease and its severity and the age, weight, physical condition and responsiveness of the mammal to be treated. Thus, it is not always possible to specify an exact “effective amount.” However, an appropriate “effective amount” in any individual case may be determined by one of ordinary skill in the art using routine experimentation.

The term “solubilizer” as used herein is intended to mean a compound that is used for dissolving the therapeutically effective amount of the active ingredient. Suitable solubilizers include non-aqueous water soluble solubilizers such as polyethylene glycols, glycerin, polyethylene glycols of various molecular weights and the like and mixtures thereof, propylene glycol, caffeine, xanthenes, gentisic acid, cyclodextrins, isopropyl alcohol and mixtures thereof. Other suitable solubilizers or co-solubilizers include polyols or amides or esters, butanediols and isomers thereof, pentaerythritol, sorbitol, mannitol, dimethyl isosorbide, polypropylene glycol, ethers of polyethylene glycols having an average molecular weight of about 200 to about 6000, such as tetrahydrofurfuryl alcohol PEG or methoxy PEG; amides such as 2-pyrrolidone, 2-piperidone, F-caprolactam, N-alkylpyrrolidone, N-hydroxyalkylpyrrolidone, N-alkylpiperidone, N-alkylcaprolactam, dimethylacetamide and the like and mixtures thereof; esters such as ethyl propionate, tributylcitrate, acetyl triethylcitrate, acetyl tributyl citrate, triethylcitrate, ethyl butyrate, triacetin, propylene glycol diacetate, .epsilon.-caprolactone and isomers thereof, delta-valerolactone and isomers thereof, beta-butyrolactone and isomers thereof, dimethyl acetamide, dimethyl isosorbide, N-methyl pyrrolidones, transcutol and the like and mixtures thereof. Examples of oils as solubilizers include mineral oil, vegetable oil, silicon oil, lanolin, refined animal oil, hydrocarbon esters derived from vegetable animal or marine origin. Useful vegetable oils include isopropyl miristate, jojoba oil, almond oil, avocado oil, coconut oil, capric-caprylic tryglyceride of fractionated coconut oil, nutmeg oil, PEG-6 apricot kernel oil (e.g., oleoyl polyoxylglycerides: Labrafil® M 1944 CS), castor oil, olive oil and oleic acid, soybean oil, sunflower oil, peanut oil, canola oil and the like and mixtures thereof. The oil may be saponifiable or unsaponifiable and liquid or solid at room temperature. Special oils are essential oils or poly unsaturated fatty acid or oils or etherified oils and modified semi-synthetic oils. An example of a semi-synthetic oil is a product of inter-esterification of hydrogenated palm oil palm kernel oil (C₈-C₁₈ triglycerides) with a melting point at about 30° C. to about 50° C.

The term “chelating agent” as used herein is intended to mean a compound which complexes with the metal ions in the formulation and avoids the unrequired/unwanted chemical reactions from occurring. Such compounds include, by way of example and without limitation, EDTA, and the like.

The term “buffering agent” as used herein is intended to mean a compound used to resist a change in pH upon dilution or addition of acid of alkali. Such compounds include, by way of example and without limitation, disodium phosphate dihydrate, potassium metaphosphate, potassium phosphate, monobasic sodium acetate and sodium citrate anhydrous and dehydrate and other such material known to those of ordinary skill in the art.

The term “emulsifier” as used herein is intended to mean a nonionic, anionic, cationic or amplioteric surfactant. Suitable emulsifiers include, but are not limited to, glyceryl stearate, polyethylene glycol 100 stearate, glyceryl monostearate, polysorbate 60, sorbitan monostearate, polyglyceryl-4 oleate, polyoxyethylene(4)lauryl ether or trivalent cationic and the like, sodium lauryl sulphate and the like and mixtures thereof.

The term “emollient” as used herein is intended to mean a compound having two actions. One is occlusive, which provides a layer of oil on the surface of the skin to slow water loss and thus increase the moisture content of the stratum corneum. The other is as a humectant, which is a substance introduced into the stratum corneum to increase its water holding capacity. Suitable emollients for use herein include, but are not limited to, alcohols, e.g., octyldodecanol, cetyl alcohol, stearyl alcohol, cetearyl alcohol, and the like; hydrocarbons, e.g., petrolatum, light mineral oil and the like; acetylated lanolin and the like and mixtures thereof.

The term “coloring agents” as used herein is intended to mean a compound capable of imparting aesthetic appeal to the pharmaceutical product or an identification mark to the product. The coloring agents used in the pharmaceutical composition herein are FDA approved colors e.g. titanium dioxide, inorganic oxides and the like.

The term “skin feel modifiers” as used herein is intended to mean a compound that assists in improving the applicability and the feel of the cream on the surface of skin. Suitable skin feel modifiers include, but are not limited to, aluminum starch octenylsuccinate and the like.

Most of these excipients are described in detail in Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### DETAILED DESCRIPTION OF THE PREFERRED EMBODIMENTS

The present invention is directed to a stable pharmaceutical composition in a semi-solid dosage form containing at least calcipotriene in an anhydrous form. In one embodiment, the anhydrous calcipotriene is in solubilized form in an amount ranging from about 0.001 to about 0.1% w/w, either in oil or a non-aqueous water soluble solvent. Suitable semisolid dosage forms include ointments, creams, gels, lotions and the like.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable-bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Preferred water-soluble ointment bases are prepared from polyethylene glycols of varying molecular weight; again, see Remington: The Science and Practice of Pharmacy for further information.

Creams, as also well known in the art, are viscous liquids or semisolid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant. Suitable emulsifiers include, but are not limited to, glyceryl stearate, polyethylene glycol 100 stearate, glyceryl monostearate, polysorbate 60, sorbitan monostearate, polyglyceryl-4 oleate, polyoxyethylene(4)lauryl ether or trivalent cationic and the like, and sodium lauryl sulphate and mixtures thereof. The cream composition can also contain a cream base that forms the major base of the composition such as polyethylene glycol-3350, polyethylene glycol-400 and also include all those compounds known to the person skilled in the art.

As will be readily understood by those skilled in the field of pharmaceutical formulation, gels are semisolid, suspension-type systems. Gel forming agent for use herein can be any gelling agent typically used in the pharmaceutical art for topical semi solid dosage forms. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® such as Carbopol 940. Also preferred are hydrophilic polymers such as polyethylene oxides, polyoxyethylene-polyoxypropylene copolymers, and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof. The amount of gelling agents varies widely and will ordinarily ranges from about 0.1% to about 2.0% by weight, based on the total weight of the composition. The gel forming agent also work by the principle of copolymerization. Under alkaline pH, carbomer in presence of water undergoes cross linking and forms a gel like structure. The degree of polymerization is dependent upon the pH. At a threshold pH, the viscosities achieved by the polymer grade is the maximum.

Lotions, are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Pastes are semisolid dosage forms in which the active agent is suspended in a suitable base. Depending on the nature of the base, pastes are divided between fatty pastes or those made from a single-phase aqueous gels. The base in a fatty paste is generally petrolatum or hydrophilic petrolatum or the like. The pastes made from single-phase aqueous gels generally incorporate carboxymethylcellulose or the like as a base.

The semisolid dosage forms of the present invention can optionally contain preservatives such as methylparaben, propylparaben and benzyl alcohol. The appropriate amount of such preservative(s) alone or in combination or any other preservatives is known to the person skilled in the art.

The semisolid dosage forms of the present invention can optionally contain antioxidants such as, for example, butylated hydroxytoluene, butylated hydroxytoluene, butylated hydroxyanisole and the like. Other antioxidants are known to the person skilled in the art.

In one embodiment of the present invention, a process for preparing a stable pharmaceutical composition in a semisolid cream dosage form involves the steps of (a) preparing an oil phase containing solubilized calcipotriene in an anhydrous form; (b) preparing an aqueous phase containing one or more buffering agents; (c) adding the oil phase of step (a) to the aqueous phase of step (b); and (d) emulsifying and homogenizing the product of step (c).

In another embodiment of the present invention, a process for preparing a stable pharmaceutical composition in a semisolid cream dosage form involves the steps of (a) preparing an oil phase containing solubilized calcipotriene in an anhydrous form; (b) preparing an aqueous phase containing one or more buffering agents and solubilized calcipotriene in an anhydrous form; (c) adding the oil phase of step (a) to the aqueous phase of step (b); and (d) emulsifying and homogenizing the product of step (c).

In yet another embodiment of the present invention, a process for preparing topical cream dosage forms involves the steps of (a) dissolving calcipotriene in propylene glycol; (b) adding ethylenediaminetetraacetic acid (EDTA) and disodium phosphate dehydrate to water; (c) adding the product of step (a) to the product of step (b) and heating the mixture to 50-55° C.; (d) forming an oil phase comprising melting polyethylene glycol-400, polyethylene glycol-3350, glyceryl stearate/PEG 100 stearate, glyceryl monostearate and octyldodecanol and maintain temperature at about 65 to about 70° C.; (e) adding the mixture of step (c) to the oil phase; (f) adding aluminum starch octenylsuccinate and titanium dioxide and homogenize for a sufficient time period, e.g., about 15 minutes; and (g) cooling under stirring to room temperature to form a stable cream composition.

One embodiment of the present invention provides a method of treating psoriasis in a human subject. Generally, the method involves topically administering the topical pharmaceutical composition of the present invention to the area of a patient in need of such treatment.

The following examples are provided to enable one skilled in the art to practice the invention and are merely illustrative of the invention. The examples should not be read as limiting the scope of the invention as defined in the claims.

### Example 1

Preparation of a topical cream dosage form. The ingredients and amounts for this example are set forth below in Table 1.

TABLE 1

| Ingredients | Concentration (% w/w) |
|---|---|
| Calcipotriene, anhydrous | 0.005 |
| Propylene Glycol | 10.00 |
| Purified Water | 2.600 |
| Edetate Disodium | 0.0065 |
| Disodium phosphate dihydrate | 0.026 |
| Polyethylene glycol-400 | 50.2625 |
| Polyethylene glycol-3350 | 18.00 |
| Glyceryl stearate/PEG 100 stearate | 5.000 |
| Glyceryl monostearate- self emulsifying | 2.500 |
| Octyldodecanol | 5.000 |
| Titanium dioxide | 0.600 |
| Aluminium starch octenylsuccinate | 6.000 |

Manufacturing Process

A. Oil Phase

1. Polyethylene glycol-400, polyethylene glycol-3350, glyceryl stearate/PEG 100 stearate, glyceryl monostearate-self emulsifying were melted, and octyldodecanol was then added to the melt. The temperature was maintained at 65 to 70° C.

B. Drug Solution Phase

1. Calcipotriene was dissolved in propylene glycol.

2. In water, EDTA and disodium phosphate dehydrate was added and dissolved.

3. The product of step (1) was added to the product of step (2) and mixed well followed by heating to 50 to 55° C., and further maintained at this temperature.

C. Emulsification

1. The drug solution phase was added to the oil phase and homogenized for 15 minutes followed by cooling under stirring to 38 to 42° C.

2. Aluminum starch octenylsuccinate and titanium dioxide was added to the product of step (1) and homogenized for 15 minutes.

3. The final composition was cooled at room temperature.

### Example 2

Preparation of a topical cream dosage form. The ingredients and amounts for this example are set forth below in Table 2.

TABLE 2

| Ingredients | Concentration (% w/w) |
|---|---|
| Calcipotriene, anhydrous | 0.005 |
| Benzyl Alcohol | 2.0 |
| Peanut Oil | 3.0 |
| Mineral Oil | 3.0 |
| Cetostearyl alcohol | 7.5 |
| White Petrolatum | 16.0 |
| Polyoxyethylene stearyl ether (Steareth 2) | 1.2 |
| Ceteth 20 | 2.5 |
| Glycerin | 3.0 |
| Disodium hydrogen phosphate, dihydrate | 0.25 |
| Sodium dihydrogen phosphate, monohydrate | 0.012 |

Manufacturing Process

A. Oil Phase

1. Mineral oil, cetostearyl alcohol, white petrolatum, steareth 2 and ceteth 20 were heated to 65 to 70° C. under stirring to until completely melted.

2. Peanut oil and benzyl alcohol were added in a container and anhydrous calcipotriene was added under stirring with continued stirring for 30 minutes to obtain a clear solution.

3. The product of step (2) was added to the product of step (1) under stirring and maintained this phase at a temperature in between 65° C. to 70° C.

B. Aqueous Phase

1. Glycerin, disodium hydrogen phosphate and sodium dihydrogen phosphate were added to purified water under stirring and heated to 65° C. to 70° C.

2. The product of step (1) was added to the oil phase (i.e., the product of step (3) of the oil phase, under stirring and homogenized for 20 minutes.

3. After completion of homogenization, the cream was cooled to room temperature under stirring.

### Example 3

The cream composition of Example 2 was compared to the innovator Dovonex cream. The cream composition of Example 2 showed a similar assay at 40° C./75% Relative Humidity to that of the Dovonex cream as seen after 3 months storage. The results are set forth below in Table 3. Preferably, the assay of the cream composition of present invention and that of Dovonex cream should fall within the range of 90 to 100% after 3 months at 40° C. and 75% Relative Humidity (RH).

TABLE 3

| | | | Assay |
|---|---|---|---|
| Composition | Duration | Storage condition | (%) |
| Example 2 of present invention | 3 months | 40° C./75% RH | 95.5 |
| Dovonex Cream: (Innovator) | 3 months | 40° C./75% RH | 92.6 |

Chromatographic Conditions:

Apparatus: A High Performance Liquid Chromatograph equipped with binary pump, variable wavelength UV detector attached with data recorder and integrator software

Column: HYPERSIL BDS C18, 150×4.6 mm, 3

Flow Rate: 1.0 millilitre/minute

Detection: UV 264 nm

Injection volume: 50 μl for assay and 100 μl for related substance

Run Time: 25 minutes for assay and 60 min for related substance

Column oven: Ambient

Retention time: About 20.0 minutes

Mobile phase: Water:Methanol (30:70) v/v

Diluent: Buffer (1.32 g Ammonium dihydrogen phosphate in 10 millilitre of water): methanol:water (3:700:297) v/v

Assay

Preparation of Standard Solution

10.0 milligram of calcipotriene was accurately weighed into a 100 millilitre volumetric flask and diluted up to the mark with diluent. 5 millilitre of the solution was pipetted out in to 250 millilitre volumetric flask and 50 millilitre tetrahydrofuran was then added and volume was made with diluent. (concentration about 2 ppm).

Preparation of Sample Solution

5 g of sample was weighed into a 25 millilitre of volumetric flask, and to it was added 10 millilitre of tetrahydrofuran. The sample was sonicated for 20 min and volume was made with tetrahydrofuran. 5.0 millilitre of this solution was pippeted out in a stoppered test tube and to it was added 20.0 millilitre of diluent. The solution was sonicated for 10 minutes, cooled in ice-cold water and filtered through a Teflon 0.45μ filter (concentration about 2 ppm).

Procedure

Equal volume of blank (diluent), five replicate injections of standard solution and sample solution prepared above were injected. Assay percentage was calculated using main peak response in sample and standard solution.

### Example 4

A cream composition, referred to as Batch 56, was prepared in substantially the same manner as in Example 2. The stability data of the cream composition was determined and showed that a single unknown maximum impurity was not present in the composition at more than 0.5% at 40° C. and 75% RH. The results are set forth below in Table 4.

TABLE 4

| | | | Related Substances | | | | |
|---|---|---|---|---|---|---|---|
| Batch | Storage | Assay | | | | Unk. | |
| No | condition | (%) | IMP B | IMPC | IMPD | Max | Total |
| 056 | Initial | 100.5 | ND | 0.12 | 0.08 | ND | 0.20 |
| 15 days | 40° C./ | 98.6 | ND | 0.11 | 0.22 | 0.06 | 0.41 |
| | 75% RH | | | | | | |
| 1 M | 40° C./ | 99.3 | 0.06 | 0.15 | 0.40 | 0.08 | 0.80 |
| | 75% RH | | | | | | |
| 2 M | 30° C./ | 99.0 | ND | 0.15 | 0.42 | 0.07 | 0.64 |
| | 65% RH | | | | | | |
| | 40° C./ | 97.1 | ND | 0.34 | 0.55 | 0.14 | 1.15 |
| | 75% RH | | | | | | |
| 3 M | 30° C./ | 98.3 | 0.03 | 0.10 | 0.69 | 0.06 | 1.00 |
| | 65% RH | | | | | | |
| | 40° C./ | 95.5 | 0.05 | 0.17 | 1.00 | 0.19 | 1.60 |
| | 75% RH | | | | | | |

For Related Substances (RS)

Preparation of Standard Solution:

Calcipotriene (about 10.0 mg) working standard was weighed into a 100 millilitre volumetric flask and diluted up to the mark with a diluent. 1 millilitre of the solution was diluted to 100 millilitre with a diluent. Further, 1 millilitre of this solution was pippeted out into a 10 millilitre volumetric flask and to it was added 1 millilitre of tetrahydrofuran and made up to volume with diluent. (concentration about 0.1 ppm).

Preparation of System Suitability Solution:

Calcipotriene Monohydrate (1 mg) was dissolved into a 10 millilitre volumetric flask with suitable dissolving means. Further, 1 millilitre of this solution was pipetted out in 10 millilitre volumetric flask and 1 millilitre of tetrahydrofuran was added to it and made up to volume with diluent.

Preparation of Sample Solution:

2 grams of sample was weighed into a 50 millilitre glass stoppered test tube and added 1 millilitre tetrahydrofuran. The solution was sonicated for 20 minutes. Next, 9 millilitre of diluent was added and sonicated for 10 minutes, cooled in ice-cold water and filtered through a Teflon 0.45μ filter and injected (concentration about 10 ppm).

Procedure:

Equal volumes of blank (diluent) system suitability solution, six replicate injections of standard solution, placebo solution and sample solution were separately injected. The % impurity using a main peak response of standard solution was calculated and the peak due to the diluent and placebo was disregarded.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention.

What is claimed is:

1. (canceled)

2. A stable pharmaceutical composition in a semisolid dosage form comprising a therapeutic effective amount of anhydrous calcipotriene in a solubilized form, wherein the calcipotriene has no more than about 0.5% of a single unknown impurity, as measured by HPLC, at 40° C. and 75% relative humidity after three months.

3. (canceled)

4. The composition of claim 2, wherein the anhydrous calcipotriene is solubilized form in oil or a non-aqueous water soluble solvent.

5. The composition of claim 4, wherein the oil is selected from a mineral oil, vegetable oil, silicon oil, lanolin, refined animal oil, hydrocarbon ester derived from a plant-derived oil, marine-derived oil or animal-derived oil and mixtures thereof.

6. The composition of claim 5, wherein the vegetable oil is selected from isopropyl myristate, jojoba oil, almond oil, avocado oil, coconut oil, capric-caprylic triglyceride of fractionated coconut oil, nutmeg oil, PEG-6 apricot kernel oil, castor oil, olive oil and oleic acid, soybean oil, sunflower oil, peanut oil, canola oil and mixtures thereof.

7. The composition of claim 4, wherein the non-aqueous water soluble solvent is selected from polyethylene glycol, propylene glycol, glycerin, caffeine, xanthene, gentisic acid, cyclodextrin, isopropyl alcohol and mixture thereof.

8. The composition of claim 4, wherein the anhydrous calcipotriene is present in a concentration of about 0.001% to about 0.1% w/w.

9. The composition of claim 8, is in the form of a cream, a gel, a lotion, or paste.

10.-12. (canceled)

13. The composition of claim 4, further comprising one or more pharmaceutically acceptable excipients.

14. (canceled)

15. A process for the preparation of a stable pharmaceutical composition in a semisolid dosage form comprising a therapeutic effective amount of anhydrous calcipotriene in a solubilized form comprising: (a) preparing an oil phase comprising solubilized calcipotriene in an anhydrous form; (b) preparing an aqueous phase comprising one or more buffering agents; (c) adding the oil phase of (a) to the aqueous phase of (b); and (d) emulsifying and homogenizing the product of (c).

16. (canceled)

17. The process of claim 15, wherein the anhydrous calcipotriene is present in a concentration of about 0.001% to about 0.1% w/w.

18. The process of claim 17, wherein the pharmaceutical composition is in the form of a cream, a gel, a lotion, or paste.

19. The process of claim 15, wherein the anhydrous calcipotriene is solubilized in oil or a non-aqueous water soluble solvent.

20. The process of claim 19, wherein the oil is selected from a mineral oil, vegetable oil, silicon oil, lanolin, refined animal oil, hydrocarbon ester derived from a plant-derived oil, marine-derived oil or animal-derived oil and mixtures thereof.

21. The process of claim 20, wherein the vegetable oil is selected from isopropyl myristate, jojoba oil, almond oil, avocado oil, coconut oil, capric-caprylic triglyceride of fractionated coconut oil, nutmeg oil, PEG-6 apricot kernel oil, castor oil, olive oil and oleic acid, soybean oil, sunflower oil, peanut oil, canola oil and mixtures thereof.

22. The process of claim 19, wherein the non-aqueous water soluble solvent is selected from a polyethylene glycol, propylene glycol, glycerin, caffeine, xanthene, gentisic acid, cyclodextrin, isopropyl alcohol and mixture thereof.

23. The method of treatment to a mammal in need thereof comprising administering a stable pharmaceutical composition in a semisolid dosage form comprising a therapeutic effective amount of anhydrous calcipotriene in a solubilized form.

24. (canceled)

25. The method of claim 23, wherein the calcipotriene has no more than about 0.5% of a single unknown impurity, as measured by HPLC, at 40° C. and 75% relative humidity after three months.

26.-37. (canceled)

38. The process of claim 15, wherein the calcipotriene has no more than about 0.5% of a single unknown impurity, as measured by HPLC, at 40° C. and 75% relative humidity after three months.
